SEQUENCE LISTING (4 sequences)

SEQ ID NO: 1 - protein, 19 aa, Artificial Sequence
  synthetic
  MISC_FEATURE (13)..(13): X=citrulline
SHQESTRGRSRGXSGRSGS

SEQ ID NO: 2 - protein, 19 aa, Artificial Sequence
  synthetic
  MISC_FEATURE (16)..(16): X=citrulline
SHQESTRGRSRGRSGXSGS

SEQ ID NO: 3 - protein, 19 aa, Artificial Sequence
  synthetic
  MISC_FEATURE (7)..(9): X=citrulline
SHQESTXGXSRGRSGRSGS

SEQ ID NO: 4 - protein, 19 aa, Artificial Sequence
  synthetic
  MISC_FEATURE (7)..(11): X=citrulline
SHQESTXGRSXGRSGRSGS

(19) United States
(12) Patent Application Publication
(10) Pub. No.: US 2021/0172946 A1
(43) Pub. Date: Jun. 10, 2021

(54) KIT FOR MEASURING ANTI-CYCLIC CITRULLINATED PEPTIDE ANTIBODY, APPLICATION THEREOF, AND TEST METHOD

(71) Applicant: JIANGSU HAOOUBO BIOPHARMACEUTICAL CO., LTD., Suzhou, Jiangsu (CN)

(72) Inventors: Lige CUI, Suzhou (CN); Le LIU, Suzhou (CN); Suqing YANG, Suzhou (CN); Ting ZHAO, Suzhou (CN); Xiaole ZHI, Suzhou (CN); Jingwen LI, Suzhou (CN); Le Xu, Suzhou (CN); Yuming Li, Suzhou (CN); Qingchun LI, Suzhou (CN)

(21) Appl. No.: 16/980,561

(22) PCT Filed: Apr. 21, 2018
(86) PCT No.: PCT/CN2018/083989

§ 371 Date: Dec. 1, 2020

(30) Foreign Application Priority Data
Mar. 13, 2018 (CN) 201810202734.6

(51) Int. Cl. G01N 33/564; G01N 33/68

(52) U.S. Cl. CPC G01N 33/564; G01N 2800/102; G01N 2440/18; G01N 33/6854

(57) ABSTRACT

A kit for measuring an anti-cyclic citrullinated peptide antibody, an application thereof, and a test method is provided. The kit includes a biotinylated antigen formed by coupling a plurality of branched peptide units having branched peptide chains using lysine, and the branched peptide units each is formed by coupling a plurality of citrullinated peptides using lysine. The kit and method have high test sensitivity and good specificity; compared with a mixed antigen, citrulline being coupled and then used as a single-component antigen, reduces the complexity of components of the antigen and the difficulty of quality control of the reagent, and improves inter-batch consistency, and quantitative test can be performed and the time needed to complete all procedures to obtain a result is 45 min.

### CROSS-REFERENCE TO RELATED APPLICATIONS

This application claims priority to PCT Application No. PCT/CN2018/083989, having a filing date of Apr. 21, 2018, which is based on Chinese Application No. 201810202734.6, having a filing date of Mar. 13, 2018, the entire contents both of which are hereby incorporated by reference.

### SEQUENCE LISTING

This application includes a separate sequence listing in compliance with the requirements of 37 C.F.R. §§ 1.824(a)(2)-1.824(a)(6) and 1.824(b), submitted under the file name “0013US01_SQL_defective_response_2-19-21”, created on Feb. 19, 2021, having a file size of 2 KB, the contents of which are hereby incorporated by reference.

### FIELD OF TECHNOLOGY

The following relates to the field of magnetic particle chemiluminescence immunodiagnosis technology, specifically relates to a kit for measuring anti-cyclic citrullinated peptide antibody, application, and test method thereof.

### BACKGROUND

Rheumatoid Arthritis (RA) is one of the most common autoimmune disease and one of the frequently-occurring chronic inflammatory joint disease, with a global incidence rate of about 1%, and the incidence rate in China is 0.32-0.36%. RA is more common in women, and the ratio of male to female is about 1:2-1:4. It is a systemic disease characterized by inflammation of the synovial membrane of the joints, which develops symmetrically from small joints to large joints, and eventually leads to joint damage and soft tissue damage in the later stages of the disease. Autoantibodies with high specificity for RA include: rheumatoid factor (RF), anti-keratin antibody (AKA), anti-perinuclear factor (APF), RA33 and anti-cyclic citrullinated peptide antibody (CCP). Rheumatoid factor is an antibody produced in the body with denatured IgG (an antibody) as the antigen due to infectious factors (bacteria, viruses, etc.), and the common rheumatoid factors include IgM, IgG, IgA and IgE types. The positive incidence in RA is 60-80%, but RF is a non-specific antibody that may exist in healthy elderly people, patients with autoimmune diseases and infectious diseases, with poor specificity.

There is also a class of specific antibodies that can be detected in the serum of patients with RA, namely anti-filaggrin autoantibodies, including anti-perinuclear factor (APF) and anti-keratin antibody (AKA). APF is a kind of antibody against keratin granules in the cytoplasm of human buccal mucosal cells and profilaggrin protein is its main target antigen. AKA is an antibody in RA serum that can react with the stratum corneum of rat esophagus and filaggrin is its main target antigen (profilaggrin is formed by connecting 10 to 12 repeating filaggrin fragments through short peptides of 7 amino acids and highly phosphorylating, it is dephosphorylated and broken from the connecting short peptides to form functional filaggrin polypeptides). These two antibodies have high specificity but low sensitivity, a negative result of APF or AKA cannot exclude RA, and studies have found that the rare amino acid citrulline in filaggrin is its main epitope. Citrulline is a non-standard amino acid and does not participate in protein translation in vivo, it is produced by treating arginine residues by arginine deiminase, and the filaggrin precursor in the keratin granules of human mucosal cells is decomposed into filaggrin subunits by an enzyme in cell differentiation, and during this process, the protein is dephosphorylated, and arginine residues are converted into citrulline residues under the enzyme catalysis. Through comparative studies, it is found that using cyclic citrullinated peptides instead of linear citrullinated peptides as the antigen substrate of ELISA can increase the sensitivity from 49% to 68%, and Anti-CCP is a new and highly specific indicator of RA.

Anti-CCP has the following three significances in the clinical application of RA:

An important indicators for early diagnosis of RA: 10% of RA can be tested positive about 10 years before the first clinical symptoms appear; 40% of RA onset and 70% of RA are positive 1 year before the first visit; early prediction of the development of undifferentiated arthritis to rheumatoid arthritis: 93% of Anti-CCP positive patients progressed to rheumatoid arthritis; 25% of Anti-CCP negative patients progressed to rheumatoid arthritis. Reduce the missed diagnosis of RF-negative patients, for 20%-57% of RA in which RF is diagnosed as being negative, Anti-CCP is positive; Anti-CCP has the same sensitivity as RF, but Anti-CCP has higher specificity (Anti-CCP: 96-100%, RF: 63%). Anti-CCP can also predict an independent index of joint damage, RA patients with positive Anti-CCP have more severe joint damage than patients with negative.

Because Anti-CCP has great clinical significance for RA, the testing technology has attracted much attention, and the development of commercial kits is very rapid.

In 1998, Schellkens et al. synthesized 31 short peptides of 9 to 19 amino acids that may contain epitopes according to the profilaggrin cDNA sequence, and replaced arginines in different parts of the molecule with citrulline, and the serum of RA patients with anti-filaggrin positive was screened by competitive ELISA. In the same peptide chain, the citrullination of different parts and the amount of citrulline all affect the affinity of antigen and antibody, indicating that citrulline is a essential ingredient for the formation of antigenic determinants.

The CCP antigen originated in 1998 and has developed into three generations now. The first-generation antigen is a synthetic linear peptide with 19 amino acid residues including citrulline, that is, citrullinated peptide, and the test sensitivity of the first generation CCP antigen is 49%, and the specificity is 96%. Later, the two serines in the nonadecapeptide were replaced with cysteine, and a disulfide bond was formed between the cysteines to form a cyclic citrullinated peptide, resulting in the second-generation CCP antigen (CCP2). The second-generation CCP antigen not only maintains a high test specificity (98%), but also has a greater sensitivity (69%). The third-generation CCP antigen is still a cyclic citrullinated peptide, but an antigenic determinant that cannot be recognized by Anti-CCP2 is added, and the test sensitivity is slightly improved (73%).

There are many kinds of citrullinated autoantigens in joint synovial fluid, and there are a large number of corresponding heterogeneous antibodies in the body fluids of patients. The citrullinated antigen or polypeptide alone cannot substantially improve the specificity and sensitivity of clinical tests. The CCP antigen currently used in the market is a single citrullinated peptide/cyclic citrullinated peptide, or a mixture of multiple citrullinated peptides/cyclic citrullinated peptides, and they are all used in the ELISA test platform, which is a qualitative test and requires manual labor, and takes a long time to get the result (about 2 hours). Although the mixed use of multiple antigens ensures high test sensitivity and specificity, it will make the reagent preparation and adjusting processes very complicated and difficult to control, resulting in large inter-batch variation of reagents. While the use of a single antigen will reduce the test sensitivity.

The Publication No. CN1796997A discloses a testing kit for diagnosing RA, a method for preparing and completing quality test standards, the testing kit comprises a box, an enzyme-labeled plate arranged in the box, a control substance and a liquid reagent, each well of the enzyme-labeled plate has a branched antigen peptide structure of the specific antigen CCP that is coated with a coating buffer and blocked by irrelevant protein or serum, and the branched antigen peptide structure takes polylysine as the core substrate and has 2 to 8 CCP branched structures, has 4 branched structures. However, the testing time of this kit is long, requiring 2 hours, and the negative coincidence rate is low.

### SUMMARY

The technical problem to be solved by the present disclosure is to provide a kit for measuring an anti-cyclic citrullinated peptide antibody with good stability, high sensitivity, good reproducibility and short testing time, and an application and a test method thereof.

To solve the above technical problems, the present disclosure employs the following technical solution:

A purpose of the present disclosure is to provide a kit for measuring an anti-cyclic citrullinated peptide antibody comprising a biotinylated antigen formed by coupling a plurality of branched peptide units having branched peptide chains using lysine, the branched peptide units each being formed by coupling a plurality of citrullinated peptides using lysine.

The citrullinated peptides are selected from the group consisting of the following amino acid sequences and combinations thereof:

SEQ ID NO. 1:
SHQESTRGRSRGXSGRSGS;
SEQ ID NO. 2:
SHQESTRGRSRGRSGXSGS;
SEQ ID NO. 3:
SHQESTXGXSRGRSGRSGS;
SEQ ID NO. 4:
SHQESTXGRSXGRSGRSGS.

A plurality of citrullinated peptides as shown in SEQ ID NO. 1 form a first branched peptide unit, a plurality of citrullinated peptides as shown in SEQ ID NO. 2 form a second branched peptide unit, a plurality of citrullinated peptides as shown in SEQ ID NO. 3 form a third branched peptide unit, a plurality of citrullinated peptides as shown in SEQ ID NO. 4 form a fourth branched peptide unit, and the biotinylated antigen comprises the first branched peptide unit, the second branched peptide unit, the third branched peptide unit, and the fourth branched peptide unit.

The lysine of the first branched peptide unit and the lysine of the second branched peptide unit are respectively coupled with one lysine, and lysine of the third branched peptide unit and the lysine of the fourth branched peptide unit are respectively coupled with another lysine.

Each of the branched peptide units respectively has 4 to 6 citrullinated peptides, and the biotinylated antigen has 4 to 6 branched peptide units.

Each of the branched peptide units respectively has 4 citrullinated peptides, and the biotinylated antigen has 4 branched peptide units.

In the biotinylated antigen, each of the citrullinated peptides is coupled with biotin.

A preparation method of the biotinylated antigen comprises the following steps:

Step 1: coupling the citrullinated peptides with lysine to form branched citrullinated peptides;

Step 2: mixing 2 to 5 mg of synthesized branched citrullinated peptides and 0.5 to 0.8 mg of biotin activated by N-hydroxysuccinimide, mixing evenly and reacting at 22 to 25° C. for 25 to 40 min;

Step 3: adding 15 to 20 μL of tris(hydroxymethyl)aminomethane buffer with a concentration of 0.04 to 0.06 mol/L, mixing and reacting at 28 to 32° C. for 15 to 30 min, then adding 550 to 650 μL of glycerol to obtain biotinylated CCP branched peptide antigen, which is stored at −20° C. for future use;

Step 4: diluting the biotinylated citrullinated branched peptide antigen into a mixed solution with a concentration of 1 to 5 μg/ml using a phosphate buffer of pH 7-7.5 and a concentration of 0.01 mol/L, to give a biotinylated antigen working fluid.

The kit further comprises magnetic particle separation reagents, alkaline phosphatase-labeled anti-human IgG antibodies, chemiluminescence substrates, calibrators, and quality control materials. Magnetic particles in the magnetic particle separation reagents have a diameter of 0.1 to 0.5 μm, have superparamagnetism, and have streptavidin groups on the surfaces thereof.

The chemiluminescence substrates used in the present disclosure is an enzyme-promoting chemiluminescence substrate using alkaline phosphatase disclosed by Patent Application No. CN201510359183.0. The chemiluminescence substrates of the present disclosure has the advantages of high intensity, high sensitivity, long duration, good stability, and the like. Due to AMPPD can act as a co-surfactant, the chemiluminescence substrate can be better combined into the chemiluminescence buffer system, thereby greatly improving the chemiluminescence efficiency, and releasing photons under the catalysis of alkaline phosphatase.

Another purpose of the present disclosure is to provide an application of the kit for testing the content of anti-cyclic citrullinated peptide antibody.

The third purpose of the present disclosure is to provide a method for testing the content of anti-cyclic citrullinated peptide antibody using the kit, which comprises the following steps: reacting a sample to be tested with a magnetic particle separation reagent and biotinylated antigen at 36 to 38° C. for 10 to 25 min to obtain a first complex, washing and then adding alkaline phosphatase-labeled anti-human IgG antibody, and reacting at 36 to 38° C. for 10 to 25 min to obtain a second complex, washing and then adding a chemiluminescent substrate and reacting at 36 to 38° C. for 5 to 10 min, and testing.

Due to the use of the above technical solutions, the present disclosure has the following advantages over the conventional art:

1. The present disclosure has high detection sensitivity, good specificity, and it is better than the products of well-known foreign manufacturers.

2. Compared with a mixed antigen, citrulline being coupled and then used as a single-component antigen, reduces the complexity of components of the antigen and the difficulty of quality control of the reagent, and greatly improves batch-to-batch consistency.

3. The present disclosure can perform quantitative detection and the time needed to complete all procedures to obtain a result is 45 min, which greatly reduces the detection time when compared with ELISA.

4. The present disclosure utilizes an alkaline phosphatase (AP)-adamantane (AMPPD) system, which is 10 times more sensitive than an ELISA test system.

5. The present disclosure is a fully automatic closed operating system with high reliability, good stability, and good reproducibility of test results.

6. The present disclosure realizes full automation from the steps of dilution, sample addition, incubation, washing, and test, which avoids result deviation caused by human operation, and effectively improves test efficiency and saves labor costs.

### BRIEF DESCRIPTION

Some of the embodiments will be described in detail, with references to the following Figures, wherein like designations denote like members, wherein:

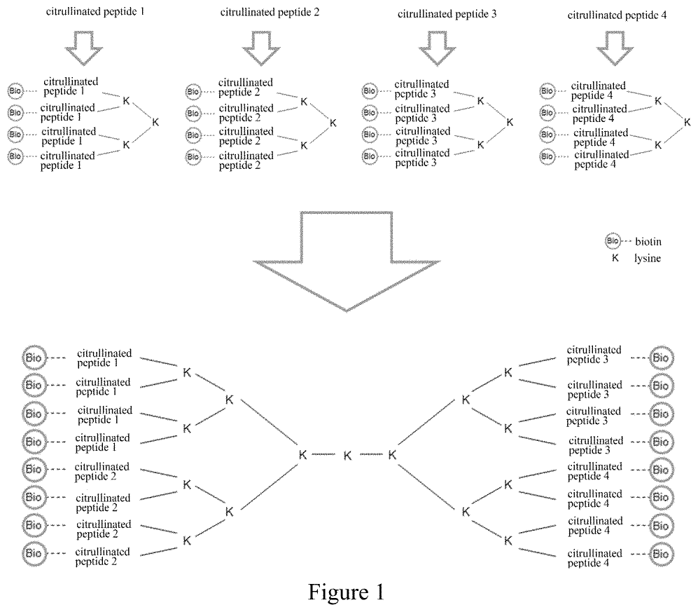

FIG. 1 is schematic structure diagrams of the respective branched peptide units and biotinylated antigen;

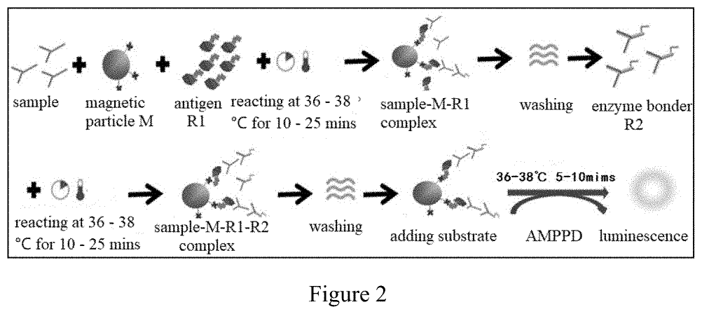

FIG. 2 is a test principle diagram; and

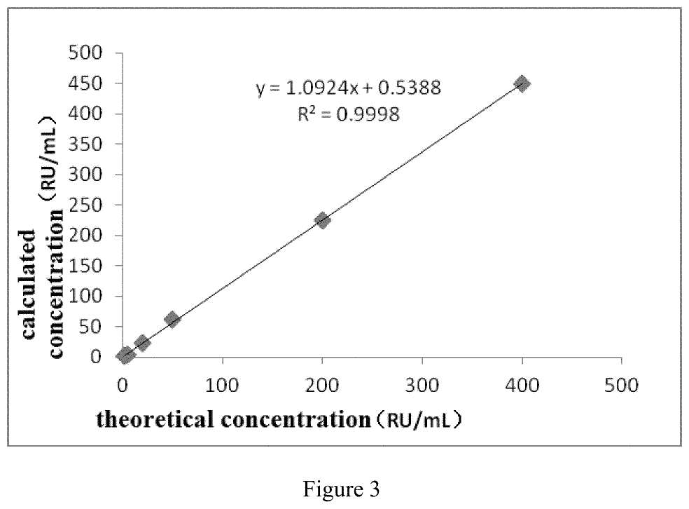

FIG. 3 is a linear regression diagram of the actual test concentration tested, according to the test method of Embodiment 6, using the test kit of anti-cyclic citrullinated peptide antibody IgG of Embodiment 5 and the theoretical concentration.

### DETAILED DESCRIPTION

In the following, the present disclosure is further explained in detail combining with specific embodiments, but the present disclosure is not limited to the following embodiments. The implementation conditions employed by the embodiments may be further adjusted according to specific requirements, undefined implementation conditions usually are conditions in conventional experiments, and the reagents in the present disclosure are all commercially available.

### Embodiment 1: Preparation of Biotinylated Antigen

### Synthesis of 16-Branched Citrullinated Peptide

According to the structural characteristics of citrulline and arginine, their spatial structure changes were predicted using bioinformatics related software, the known citrullinated antigens were analyzed, classified to screen out a citrulline-containing polypeptide library. The citrulline-containing polypeptide library and rheumatoid arthritis serum were screened to obtain a group of citrullinated peptides from which four citrullinated peptides with the highest combined test sensitivity were screened. Utilizing the characteristics that a lysine has two amino groups, the citrullinated peptides were coupled into a molecule with four identical branched peptide chains, and finally four kinds of four-branched citrullinated peptides were obtained. Then these four different branched peptide molecules were coupled into one molecule with lysine, which was 16-branched citrullinated peptide, wherein the method of coupling the branched peptides with lysine was a conventional method.

The amino acid sequences of citrulline selected are:

SEQ ID NO. 1: SHQESTRGRSRGXSGRSGS, namely Ser His Gln Glu Ser Thr Arg Gly Arg Ser Arg Gly Xaa Ser Gly Arg Ser Gly Ser, wherein, X and Xaa respectively represent citrulline, as shown in Sequence 1 of the sequence listing;

SEQ ID NO. 2: SHQESTRGRSRGRSGXSGS, namely Ser His Gln Glu Ser Thr Arg Gly Arg Ser Arg Gly Arg Ser Gly Xaa Ser Gly Ser, wherein, X and Xaa respectively represent citrulline, as shown in Sequence 2 of the sequence listing;

SEQ ID NO. 3: SHQESTXGXSRGRSGRSGS, namely Ser His Gln Glu Ser Thr Xaa Gly Xaa Ser Arg Gly Arg Ser Gly Arg Ser Gly Ser, wherein, X and Xaa respectively represent citrulline, as shown in Sequence 3 of the sequence listing;

SEQ ID NO. 4: SHQESTXGRSXGRSGRSGS, namely Ser His Gln Glu Ser Thr Xaa Gly Arg Ser Xaa Gly Arg Ser Gly Arg Ser Gly Ser, wherein, X and Xaa respectively represent citrulline, as shown in Sequence 4 of the sequence listing;

The specific preparation method of the biotinylated antigen:

### I. Materials and Instruments:

Materials: synthesized 16-branched citrullinated peptides, biotin activated by N-hydroxysuccinimide, tris(hydroxymethyl)aminomethane buffer, glycerol, and phosphate buffer;

Instruments: a reagent cryopreservation box, and a biochemical incubator.

### II. Preparation Steps:

Step 1: 2 mg of synthesized 16-branched citrullinated peptides and 0.5 mg of biotin activated by N-hydroxysuccinimide were mixed evenly and reacted at 25° C. for 30 min;

Step 2: 20 μL of tris(hydroxymethyl)aminomethane buffer with a concentration of 0.05 mol/L was added, mixed and reacted at 30° C. for 30 min, then 600 μL of glycerol were added to obtain biotinylated CCP branched peptide antigen, which was stored at −20° C. for future use;

Step 3: The biotinylated citrullinated branched peptide antigen was diluted into a mixed solution with a concentration of 2 μg/ml using a phosphate buffer of pH 7.5 and a concentration of 0.01 mol/L, to give a biotinylated antigen working fluid.

The structure of the biotinylated antigen is shown in FIG. 1, wherein, citrullinated peptide 1 is citrullinated peptide shown in SEQ ID NO. 1, citrullinated peptide 2 is citrullinated peptide shown in SEQ ID NO. 2, citrullinated peptide 3 is citrullinated peptide shown in SEQ ID NO. 3, and citrullinated peptide 4 is citrullinated peptide shown in SEQ ID NO. 4.

### Embodiment 2: Preparation of Alkaline Phosphatase-Labeled Anti-Human IgG Antibody

### Materials and Instruments:

Materials: anti-human IgG antibody, alkaline phosphatase, coupling reagent 2-iminothiolane hydrochloride, glycine, and tris(hydroxymethyl)aminomethane buffer.

Instruments: a G-25 gel column, a reagent cryopreservation box, a Supperdex200 gel purification column, an analytical balance, and a biochemical incubator.

### Operation Steps:

Step 1: 3 mg of anti-human IgG antibody was added into 40 mL of a coupling reagent 2-iminothiolane hydrochloride with a concentration of 10 mg/mL, and the system stood at 20° C. for 20 min;

Step 2: 2 mL of 0.08 mol/L glycine solution was added, the system stood at 20° C. for 4 min and was desalted through the G-25 gel column, and the activated anti-human IgG antibody was collected and stored at 5° C. for future use;

Step 3: 3 mg of an alkaline phosphatase solution was added into 4 mg/mL N-succinimidyl 4-(maleimidomethyl)cyclohexane-1-carboxylate solution, the system stood at 25° C. for 30 min and was desalted through the G-25 gel column, and the activated alkaline phosphatase was collected and stored at 5° C. for future use;

Step 4: The activated anti-human IgG antibody and the activated alkaline phosphatase were mixed, and the mixture stood at 5° C. for 20 h, purified through Supperdex200 gel purification column to obtain a concentrated connected material solution, which was stored at 5° C. for future use;

Step 5: The concentrated connected material solution of Step 4 was diluted into a concentration of 1 μg/mL of the alkaline phosphatase-labeled anti-human IgG antibody using a mass ratio of 1% of bovine serum albumin and tris(hydroxymethyl)aminomethane buffer of pH 7.8 to 8.0 and a concentration of 0.05 mol/L, namely the alkaline phosphatase-labeled anti-human IgG antibody.

### Embodiment 3: Preparation of Calibrator: for Calibration Curve

### I. Materials and Instruments:

Materials: anti-citrullinated peptide antibody, phosphate buffer, and standard substance;

### II. Preparation Steps:

The anti-citrullinated peptide antibody was selected, diluted according to a certain proportion using a phosphate buffer of pH 7.5 and a concentration of 0.01 mol/L, and with reference to the standard substance, prepared to be calibrators respectively with concentrations of 20 RU/ml and 200 RU/ml.

### Embodiment 4: Preparation of Quality Control Materials

### I. Materials and Instruments:

Materials: anti-citrullinated peptide antibody, phosphate buffer, and standard substance;

### II. Preparation Steps:

The anti-citrullinated peptide antibody was selected, diluted according to a certain proportion using a phosphate buffer of pH 7.5 and a concentration of 0.01 mol/L, and with reference to the standard substance, prepared to be quality control materials respectively with concentrations of 10 RU/ml and 80 RU/ml.

### Embodiment 5: Test Kit of Anti-Cyclic Citrullinated Peptide Antibody IgG

A test kit of anti-cyclic citrullinated peptide antibody IgG of the present embodiment, comprises:

biotinylated antigen (concentration: 2 μg/mL) prepared according to the method of Embodiment 1, 5 mL;

alkaline phosphatase-labeled anti-human IgG antibody (concentration: 1 μg/mL) prepared according to the method of Embodiment 2, 30 mL;

magnetic particle separation reagent purchased from Thermo Fisher, 5 mL;

calibrator prepared according to the method of Embodiment 3, 1 mL;

quality control material prepared according to the method of Embodiment 4, 1 mL;

chemiluminescence substrates prepared according to Formula II of APSH-1 in Table 1.2 disclosed in Patent Application No. CN201510359183.0, an enzyme-promoting chemiluminescence substrate using alkaline phosphatase, namely:

| | | |
|---|---|---|
| 2-amino-2-methyl-1-propanol | 200 | mM |
| AMPPD | 0.5 | g/L |
| HOBEP-1 | 0.5 | g/L |
| mgSO₄ | 5.0 | Mm |
| ZnCl₂ | 1.0 | mM |
| ProClin300 | 0.1 | g/L. |

### Embodiment 6: Test of Kit

The kit of Embodiment 6 adopts an automatic chemiluminescence analyzer for testing, which specifically comprises the following steps:

Step 1: the test kit of Embodiment 5 was used in conjunction with a suitable automatic chemiluminescence analyzer. The kit was placed in the corresponding position of the reagent storehouse of the automatic chemiluminescence analyzer, and the kit information was input into the instrument system through the barcode scanner or set through the instrument supporting software;

Step 2: the calibrator was placed in the sample storehouse of the instrument, scanned through the barcode scanner to identify the calibrator information, and assigned to the position of the calibrator in the instrument system;

Step 3: the quality control material/sample to be tested were placed in the sample storehouse of the instrument, and the corresponding test information was edited through the instrument supporting software;

Step 4: start running program, and all the processing steps of the calibrator/quality control material/sample to be tested were automatically performed, wherein, the sample to be tested and magnetic particle separation reagent as well as biotinylated antigen were reacted at 37° C. for 15 min to obtain a first complex, the first complex was washed and the alkaline phosphatase-labeled anti-human IgG antibody was added, and the system was reacted at 37° C. for 15 min to obtain a second complex, the second complex was washed and then the chemiluminescent substrate was added, and the system was reacted at 37° C. for 5 min, and tested.

When the test kit is used in conjunction with an automatic chemiluminescence analyzer, the steps of dilution, sample addition, incubation, washing, and testing are fully automated, and unattended flow operation is possible. The fully automatic closed operating system not only has simple and convenient operation, high reliability, good stability, and good reproducibility of test results, but also avoids result deviation caused by human operation, and effectively improves test efficiency and saves labor costs.

### Embodiment 7: Implementation of Test and Evaluation of Test Effect

(1) Sample Comparison

Negative and positive coincidence rate: The test kit of Embodiment 5 of the present disclosure was used to test the content of CCP in 250 clinical sera, and compared with the CCP antibody ELISA test kit of EURO DIAGNOSTICA company (Table 1). The results show that, compared with the ELISA test kit, the negative coincidence rate of the CCP kit of the present disclosure is 99.0% (198/200), and the positive coincidence rate is 94.0% (48/50), indicating that the kit and the existing anti-CCP antibodies testing reagents on the market have a high degree of consistency. The data are shown in Table 1.

TABLE 1
Sample comparison

| | | Kit of Embodiment 5 | | |
|---|---|---|---|---|
| Clinic Sample | | Negative | Positive | Total |
| ELISA | Negative | 198 | 2 | 200 |
| testing kits | Positive | 2 | 48 | 50 |
| | Total | 200 | 50 | 250 |

(2) Sensitivity: The LOD of the test kit of Embodiment 5 of the present disclosure is 0.039 RU/ml, while the sensitivity of the CCP antibody ELISA test kit of EURO DIAGNOSTICA is 2 RU/ml. The kit of Embodiment 5 of the present disclosure was used to test the minimum detectable quantity reference substances (L1, L2, L3) of the testing company, and the test was repeated 3 times. The operation was according to the kit instructions and the test results was evaluated.

L1 reference substance concentration: the concentration was 60 RU/ml, the concentration error was not more than 10%;

L2 reference substance concentration: the concentration was 40 RU/ml, the concentration error was not more than 10%;

L3 reference substance concentration: the concentration was 10 RU/ml, the concentration error was not more than 10%.

(3) Linearity: a high-value serum (theoretical concentration 400 RU/ml, measured concentration 449.4 RU/ml) was diluted according to 1/1, 1/2, 1/8, 1/20, 1/80, 1/200, the diluted samples were tested through the kit of Embodiment 5 of the present disclosure, and a regression curve based on the dilution ratio and the detection concentration was drawn. The squared value of the correlation coefficient R was found out. The results are shown in FIG. 3, which shows that the linear correlation coefficient of the CCP kit is 0.9998, the result is greater than 0.9900, and the slope is 1.0924.

(4) Accuracy: the accuracy of the kit of Embodiment 5 of the present disclosure is evaluated through sample recovery. A high-value serum H (measured concentration is about 300 RU/ml), a median-value serum M (measured concentration is about 100 RU/ml), and a low-value serum L (measured concentration is about 50 RU/ml)) were added to the corresponding 3 parts of basal serum (measured concentration <10 RU/ml) according to 1:9, and the concentrations were calculated. Results: The sample recovery of serum was between 85% and 115%. The data are shown in Table 2.

Note: Sample recovery=Measured concentration of sample after addition/(0.1*Measured concentration of Sample A+0.9*Measured concentration of Sample B)*100%

TABLE 2
Accuracy

| | Measured | | Measured | Measured | | |
|---|---|---|---|---|---|---|
| | concen- | | concen- | concen- | | |
| | tration | | tration | tration | | Average |
| | of | | of | of | | of |
| Sample | Sample | Sample | Sample | sample | Sample | sample |
| A | A | B | B | after | recovery | recovery |
| High- | 281.87 | Basal | 4.03 | 37.65 | 111.97% | 102.75% |
| value | 290.99 | serum | 5.14 | 34.14 | 98.59% | |
| serum | 298.45 | 1 | 4.27 | 33.06 | 97.69% | |
| H | | | | | | |
| Median- | 102.04 | Basal | 7.28 | 18.59 | 112.30% | 108.55% |
| value | 105.25 | serum | 6.40 | 17.50 | 111.06% | |
| serum | 106.52 | 2 | 6.66 | 16.36 | 102.28% | |
| M | | | | | | |
| Low- | 44.01 | Basal | 5.49 | 9.15 | 92.06% | 96.97% |
| value | 53.79 | serum | 5.39 | 9.42 | 95.60% | |
| serum | 57.67 | 3 | 4.40 | 9.25 | 103.25% | |
| L | | | | | | |

(5) Precision: the test kit of Embodiment 5 of the present disclosure was used to test the quality control materials at three concentrations, twice a day in the morning and afternoon, with 4 repetitions each time for a total of 10 days, a total of 80 measurements for each concentration were made to calculate the coefficient of variation (coefficient of variation CV=concentration mean/standard deviation*100%). The results show that the coefficient of variation is within 10%.

TABLE 3
Precision

| Concentration | Test | Inter-assay |
|---|---|---|
| (RU/ml) | repetition | CV (%) |
| 10 | 80 | 5.2 |
| 20 | 80 | 3.1 |
| 100 | 80 | 6.4 |

(6) Stability: the kit of Embodiment 5 of the present disclosure was placed at 4° C. for 7 days and 37° C. for 7 days, respectively, and measured the quality control signal retention rate of 3 kinds of concentration, namely high concentration (measured concentration was about 200 RU/ml), medium concentration (measured concentration was about 80 RU/ml), and low concentration (measured concentration was about 10 RU/ml). The results were all >90%, indicating that the kit is stable and meets clinical requirements. The data are shown in Table 4.

TABLE 4
Stability

| | | | | | | | Re- |
|---|---|---|---|---|---|---|---|
| Sam- | 4°C. D7 | | | 37° C. D7 | | | tention |
| ple | RLU | AVER | CV | RLU | AVER | CV | rate |
| 10 | 125347 | 128684 | 3.67% | 124066 | 123823 | 0.28% | 96.22% |
| RU/ | 132020 | | | 123580 | | | |
| ml | | | | | | | |
| 80 | 932437 | 889415 | 6.84% | 916876 | 901594 | 2.40% | 101.37% |
| RU/ | 846392 | | | 886311 | | | |
| ml | | | | | | | |
| 200 | 2377448 | 2398137 | 1.22% | 2316782 | 2380701 | 3.80% | 99.27% |
| RU/ | 2418826 | | | 2444620 | | | |
| ml | | | | | | | |

(7) Specificity: different concentrations of bilirubin, hemoglobin, rheumatoid factor, triglyceride, and human anti-mouse antibody were added to serum with different concentrations, namely high concentration (300 RU/ml), medium concentration (100 RU/ml) and low concentration (20 RU/ml), and the test results showed: the added substances had no effect on the test result of the kit of Embodiment 5 of the present disclosure. The data are shown in Table 5.

TABLE 5
Specificity results

| Interferent | Addition concentration | | Cross reaction rate (%) |
|---|---|---|---|
| Bilirubin | 20 | mg/dL | 0.89 |
| Hemoglobin | 1000 | mg/dL | 1.52 |
| Triglyceride | 2000 | mg/dL | 3.01 |
| Human anti-mouse | 2000 | ng/mL | 2.64 |
| Rheumatoid factor | 1000 | IU/mL | 1.86 |

The results in Table 5 show that the above added substances have no effect on the test results of the anti-cyclic citrullinated peptide antibody test kit of Embodiment 5.

(8) Inter-batch variation: three different batches of the kits of Embodiment 5 of the present disclosure were used to test the internal precision reference substances of Haooubo (RP1:10 RU/ml, RP2: 20 RU/ml, RP3: 100 RU/ml), each with 10 repetitions, the mean (M), standard deviation (SD) and inter-batch coefficient of variation (CV) of the 30 test results were calculated, and the results should meet the requirements CV<15%. The calculation formula is:

$$CV = \frac{SD}{M} \times 100 \%$$

wherein:

CV—Coefficient of variation;

SD—Standard deviation of 30 test results

M—Mean of 30 test results

TABLE 6
Inter-batch variation

| | | RP1: | RP2: | RP3: |
|---|---|---|---|---|
| | | 10 RU/ml | 20 RU/ml | 100 RU/ml |
| LOT | Name | Dose | Dose | Dose |
| 1 | 1 | 10.68 | 20.19 | 105.25 |
| | 2 | 10.54 | 20.20 | 104.33 |
| | 3 | 10.77 | 19.42 | 100.15 |
| | 4 | 9.79 | 21.62 | 100.19 |
| | 5 | 9.79 | 20.03 | 96.84 |
| | 6 | 9.91 | 20.37 | 90.88 |
| | 7 | 9.64 | 18.31 | 92.58 |
| | 8 | 9.49 | 20.60 | 87.30 |
| | 9 | 10.51 | 20.03 | 94.73 |
| | 10 | 10.35 | 20.78 | 99.34 |
| 2 | 11 | 11.25 | 22.69 | 106.32 |
| | 12 | 10.96 | 23.48 | 111.36 |
| | 13 | 10.11 | 20.37 | 104.09 |
| | 14 | 10.45 | 21.90 | 103.03 |
| | 15 | 9.98 | 20.12 | 105.74 |
| | 16 | 10.20 | 20.60 | 104.41 |
| | 17 | 11.72 | 24.12 | 109.19 |
| | 18 | 11.36 | 20.20 | 119.89 |
| | 19 | 10.93 | 22.55 | 99.78 |
| | 20 | 11.30 | 23.73 | 99.68 |
| 3 | 21 | 11.90 | 21.55 | 101.03 |
| | 22 | 9.99 | 22.01 | 90.05 |
| | 23 | 10.59 | 21.12 | 101.15 |
| | 24 | 10.23 | 22.13 | 110.73 |
| | 25 | 9.58 | 20.84 | 99.34 |
| | 26 | 9.32 | 21.33 | 92.99 |
| | 27 | 9.98 | 19.61 | 98.38 |
| | 28 | 10.16 | 21.41 | 97.99 |
| | 29 | 10.04 | 18.75 | 90.63 |
| | 30 | 10.28 | 20.83 | 93.20 |

| | | | |
|---|---|---|---|
| AVER | 10.39 | 21.03 | 100.35 |
| SD | 0.65 | 1.38 | 7.23 |
| CV | 6.30% | 6.56% | 7.20% |

It can be seen from Table 6 that the inter-batch variation is less than 10%, and the inter-batch variation is small, which meets the industry requirements.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A kit for measuring an anti-cyclic citrullinated peptide antibody, comprising a biotinylated antigen formed by coupling a plurality of branched peptide units having branched peptide chains using lysine, wherein each of the branched peptide units are formed by coupling a plurality of citrullinated peptides using lysine.

2. The kit for measuring an anti-cyclic citrullinated peptide antibody according to claim 1, wherein the citrullinated peptides are selected from the group consisting of the following amino acid sequences and combinations thereof:
SEQ ID NO. 1:
SHQESTRGRSRGXSGRSGS;
SEQ ID NO. 2:
SHQESTRGRSRGRSGXSGS;
SEQ ID NO. 3:
SHQESTXGXSRGRSGRSGS;
and
SEQ ID NO. 4:
SHQESTXGRSXGRSGRSGS.

3. The kit for measuring an anti-cyclic citrullinated peptide antibody according to claim 2, wherein a plurality of citrullinated peptides as shown in SEQ ID NO. 1 form a first branched peptide unit, a plurality of citrullinated peptides as shown in SEQ ID NO. 2 form a second branched peptide unit, a plurality of citrullinated peptides as shown in SEQ ID NO. 3 form a third branched peptide unit, a plurality of citrullinated peptides as shown in SEQ ID NO. 4 form a fourth branched peptide unit, and the biotinylated antigen comprises the first branched peptide unit, the second branched peptide unit, the third branched peptide unit, and the fourth branched peptide unit.

4. The kit for measuring an anti-cyclic citrullinated peptide antibody according to claim 3, wherein the lysine of the first branched peptide unit and the lysine of the second branched peptide unit are respectively coupled with one lysine, and the lysine of the third branched peptide unit and the lysine of the fourth branched peptide unit are respectively coupled with another lysine.

5. The kit for measuring an anti-cyclic citrullinated peptide antibody according to claim 1, wherein each of the branched peptide units respectively has 4 to 6 citrullinated peptides, and the biotinylated antigen has 4 to 6 branched peptide units.

6. The kit for measuring an anti-cyclic citrullinated peptide antibody according to claim 1, wherein in the biotinylated antigen, each of the citrullinated peptides is coupled with biotin.

7. The kit for measuring an anti-cyclic citrullinated peptide antibody according to claim 1, wherein the kit further comprises a magnetic particle separation reagent, an alkaline phosphatase-labeled anti-human IgG antibody, and a chemiluminescence substrate.

8. The kit for measuring an anti-cyclic citrullinated peptide antibody according to claim 7, wherein magnetic particles in the magnetic particle separation reagents have a diameter of 0.1 to 0.5 μm, have superparamagnetism, and have streptavidin groups on surfaces thereof.

9. The kit for measuring an anti-cyclic citrullinated peptide antibody according to claim 1, wherein the biotinylated antigen is prepared by the following steps:
Step 1: coupling the citrullinated peptides with lysine to form branched citrullinated peptides;
Step 2: mixing 2 to 5 mg of synthesized branched citrullinated peptides and 0.5 to 0.8 mg of biotin activated by N-hydroxysuccinimide evenly, and reacting at 22 to 25° C. for 25 to 40 min;
Step 3: adding 15 to 20 μL of tris(hydroxymethyl)aminomethane buffer with a concentration of 0.04 to 0.06 mol/L, mixing and reacting at 28 to 32° C. for 15 to 30 min, then adding 550 to 650 μL of glycerol to obtain biotinylated CCP branched peptide antigen, which is stored at −20° C. for future use; and
Step 4: diluting the biotinylated citrullinated branched peptide antigen into a mixed solution with a concentration of 1 to 5 μg/ml using a phosphate buffer of pH 7-7.5 and a concentration of 0.01 mol/L, to give a biotinylated antigen working fluid.

10. An application of the kit according to claim 1 for testing the content of anti-cyclic citrullinated peptide antibody.

11. A method for testing the content of anti-cyclic citrullinated peptide antibody using the kit according to claim 1, the method comprising: reacting a sample to be tested with a magnetic particle separation reagent and biotinylated antigen at 36 to 38° C. for 10 to 25 min to obtain a first complex, washing and then adding alkaline phosphatase-labeled anti-human IgG antibody, and reacting at 36 to 38° C. for 10 to 25 min to obtain a second complex, washing and then adding a chemiluminescent substrate and reacting at 36 to 38° C. for 5 to 10 min, and testing.
